(19) United States
(12) Patent Application Publication
(10) Pub. No.: US 2022/0093225 A1
(43) Pub. Date: Mar. 24, 2022

(54) System and Method for Patient Data Provision, Consumption, and Royalty Processing

(71) Applicant: Electronic Health Record Data, Inc., Fort Worth, TX (US)

(72) Inventors: Ronald Raymond Austring, English Harbour (AG); Kenneth A. Hill, SR., Fort Worth, TX (US)

(73) Assignee: .Electronic Health Record Data, Inc., Fort Worth, TX (US)

(21) Appl. No.: 17/104,603

(22) Filed: Nov. 25, 2020

Related U.S. Application Data
(60) Provisional application No. 63/080,412, filed on Sep. 18, 2020.

(51) Int. Cl. G16H 10/60; G16H 10/40; G06F 21/62; G06Q 20/08

(52) U.S. Cl. CPC G16H 10/60; G06Q 20/0855; G06F 21/6245; G16H 10/40

(57) ABSTRACT

A patient data provision, consumption, and royalty processing system is disclosed that can facilitate patient data distribution and royalty payment. An EHR Data Utility can transfer patient clinical and pharmaceutical data from an EPOR database from data providers to data consumers with real-time (sub-millisecond delay), secure access. Only the data requested can be provided with each request. The present disclosure can facilitate settlement between the data consumer and the data provider that can result in the exchange of digital currency (Bitcoin SV), cryptocurrency (e.g., Bitcoin®, etc.), utility tokens (such as EHRCash™), vouchers, wire transfers, ACH transactions, or other suitable currency, between the parties involved in the patient data request and provisioning. The present disclosure can eliminate the need for standard accounting processes (e.g., invoicing, statements, accounts receivable and accounts payable) in order to transfer money between data exchange partners.

### CROSS REFERENCE TO RELATED APPLICATIONS

The present application claims priority to U.S. Provisional Application Ser. No. 63/080,412, filed on Sep. 18, 2020, and entitled “PATIENT DATA PROVISION, CONSUMPTION, AND ROYALTY PROCESSING SYSTEM,” the entirety of which is incorporated herein by reference.

### TECHNICAL FIELD

The present disclosure generally relates to patient data processing systems, and more specifically to patient data processing systems configured to facilitate patient data transactions and currency exchange between multiple entities.

### BACKGROUND

Numerous healthcare providers participate in each phase of care. Healthcare service providers, including pharmacies, physicians, hospitals, laboratories, clinics, medical institutions, and regulatory agencies that develop clinical standards of care, historically have operated their own data systems, typically according to unique and different formats and processes.

A network of three database components aggregate and maintain patient data from a plurality of sources. The three database components include a structured healthcare database called electronic patient outcome data (“EPO data”); an electronic pharmacy record (“ePR”) database; and an electronic patient outcome record (“EPOR”) database that is populated by data from the EPO data and ePR databases.

A patient's private information, including the patient's name, address, phone number, social security number, insurance information, medical history, clinical information, and other relevant information, can be stored in an Electronic Health Record (EHR) database, such as an Electronic Patient Outcome Record (EPOR), Solid POD, XML file, or other suitable data storage element.

Multiomics can tailor drugs to fit a patient's complex human structure and can deliver better patient outcomes. Optimal use of this data can require collection before, during and after drug therapy. Data queries, designed for drug research, can cross reference this data with the actual prescribed usage of the drugs, resulting in an extremely valuable feedback loop to the manufacturer.

An essential participant in healthcare services is another entity, the “payer” often an insurer, who pays for the services rendered and the claims made by the other healthcare providers. The insurance system includes the insurance companies that provide funds for payments to providers and to intermediaries that submit, adjudicate and facilitate payment transactions for services rendered to patients.

### SUMMARY

The present disclosure achieves technical advantages as a system and method for patient data provision, consumption, and royalty processing providing patient data distribution and payment.

The present disclosure solves the technological problem of identifying relevant electronic patient data implemented over multiple disparate systems that typically do not communicate with each other. Additionally, the present disclosure overcomes issues related to the calculation of royalties using one or more parameters based on the patient data. Further, the present disclosure solves the problems associated with incentivizing data providers to provide patient data to data consumers to improve patient outcomes.

In particular, the present disclosure improves the performance of traditional systems by facilitate settlement between the data consumer and the data provider that can result in the exchange of a digital currency (Bitcoin SV), cryptocurrency (e.g., Bitcoin®, etc.), utility tokens (e.g., EHRCash™, vouchers, wire transfers. ACH transactions, or other suitable currency, between the parties involved in the patient data request and provisioning. The present disclosure can eliminate the need for standard accounting processes (e.g., invoicing, statements, accounts receivable and accounts payable) in order to transfer money between data exchange partners. Advantageously, the present disclosure can implement an EHR data blockchain system (including an EHR Data API and an EHR transaction blockchain) that can provide a centralized patient data processing location that creates transparency, immutable verification, interactive record editing, and cryptocurrency transactions. The EHR Data API can access and retrieve patient records and generate metadata to rectify errors in a patient's record. The EHR patient transaction blockchain API can be configured to store the patient data, as well as execute smart contracts, and control the execution of cryptocurrency transfers, among other functions.

It is an object of the disclosure to provide an EHR Data Utility for transferring patient clinical and pharmaceutical data from the EPOR from data providers to data consumers with real-time (sub-millisecond delay), secure access. It is another object of the disclosure to provide only the data requested and not a patient's entire patient data record with each request. It is another object of the invention to effect payment from the data consumer to the data provider for the patient data provided.

In one exemplary embodiment, a method of providing real-time patient data sale processing, includes the steps of: generating, via a processor, a global patient record for a first patient; receiving a query request having one or more parameters for patient data from a data consumer over an encrypted network; correlating, via an EHR Data utility, the one or more parameters with a plurality of global patient records stored in a database to find matching patient data; returning the matching patient data from one or more data providers to the data consumer; and paying a royalty to the one or more data providers, the utility and the first patient. Wherein the query parameters include demographic data. Wherein the query parameters include pharmaceutical data. Wherein the query parameters include laboratory data. Wherein the query parameters include mutiomic data. Wherein the query request is stored in a distributed ledger. Further comprising automatically identifying the recipients of a payment request by storing a list of the parties that provide patient data included in the matching patient data. Wherein the royalty is a flat-fee processing fee paid to all parties involved in the query transaction. Wherein a royalty percentage is calculated based upon the amount of data contributed. Wherein the paying of royalties is executed via a smart contract on a blockchain.

In another exemplary embodiment, a method of providing ad-hoc patient data purchase processing, includes the steps of: storing patient-related data received from a data provider in a global patient record database; receiving a data sale request, having one or more data sales criteria, from a data consumer; querying the database for patient data matching the data sales criteria; generating a data sale result set having patient data that matches the data sale request; and facilitating settlement between the data consumer and the data provider by paying a royalty to the parties that contributed data to the data sale result set. Wherein the query parameters include demographic data. Wherein the query parameters include pharmaceutical data. Wherein the query parameters include laboratory data. Wherein the query parameters include mutiomic data. Wherein the query request is stored in a distributed ledger. Further comprising automatically identifying the recipients of a payment request by storing a list of the parties that provide patient data included in the matching patient data. Wherein the royalty is a flat-fee processing fee paid to all parties involved in the query transaction. Wherein a royalty percentage is calculated based upon the amount of data contributed. Wherein the paying of royalties is executed via a smart contract on a blockchain.

### BRIEF DESCRIPTION OF THE DRAWINGS

FIG. 1 illustrates a schematic view of an EHR data utility system, in accordance with one or more exemplary embodiments of the present disclosure;

FIG. 2 illustrates a schematic view of an EHR data utility architecture, in accordance with one or more exemplary embodiments of the present disclosure;

FIG. 3 illustrates a flowchart diagram exemplifying control logic embodying features of a method of real-time patient data sale processing, in accordance with one or more exemplary embodiments of the present disclosure; and

FIG. 4 illustrates a flowchart diagram exemplifying control logic embodying features of a method of ad-hoc patient data purchase processing, in accordance with one or more exemplary embodiments of the present disclosure.

### DETAILED DESCRIPTION OF THE INVENTION

The preferred version of the disclosure presented in the following written description and the various features and advantageous details thereof, are explained more fully with reference to the non-limiting examples included in the accompanying drawings and as detailed in the description, which follows. Descriptions of well-known components have been omitted so to not unnecessarily obscure the principle features described herein. The examples used in the following description are intended to facilitate an understanding of the ways in which the disclosure can be implemented and practiced. Accordingly, these examples should not be construed as limiting the scope of the claims.

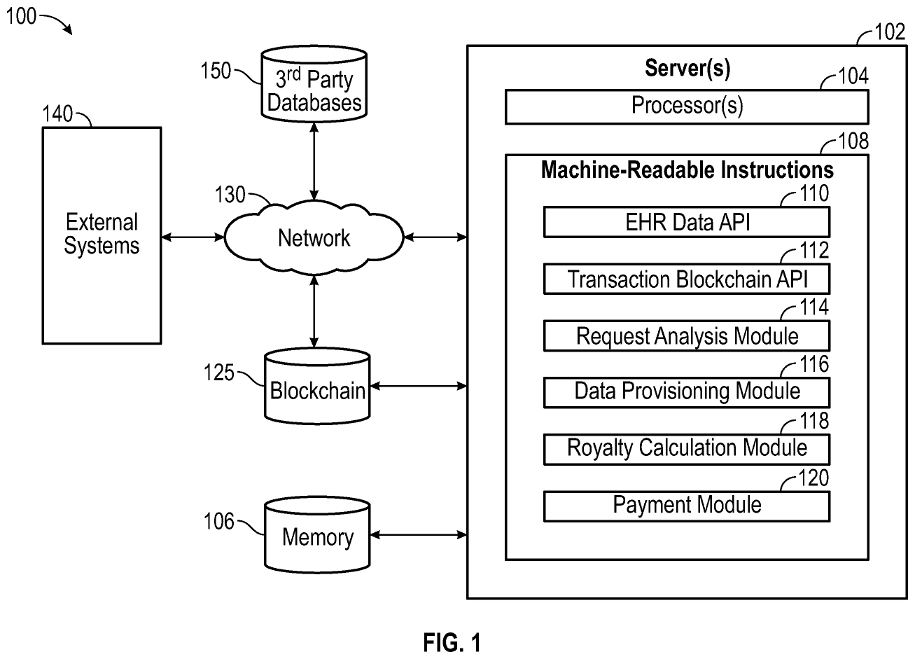

FIG. 1 illustrates an Electronic Health Record (EHR) data utility system 100, in accordance with one or more exemplary embodiments of the present disclosure. The EHR-Data-Utility system 100 can include a server 102, third party databases 106, a distributed ledger 125, a network 130, and external systems 140.

The server 102 can include one or more processors (or cores) 104, a memory 106, and machine-readable instructions 108. In one exemplary embodiment, the machine-readable instructions 108 can include an EHR Data API 110, a transaction blockchain API 112, a request analysis module 114, a data provisioning module 116, a royalty calculation module 118, and a payment module 120. In another exemplary embodiment, the server 102 can host an EHR Data Portal that can provide a DP or DC user (e.g., Physician, Manufacturer, Pharmacy. or other relevant entity) with access to the system 100 after an authenticated login. In one exemplary embodiment, the EHR Data Portal can be achieved via software, hardware, an application programming interface (API), a network connection, a network transfer protocol, HTML, DHTML, JavaScript, Dojo, Ruby, Rails, other suitable applications, or suitable combinations thereof. In another exemplary embodiment, the EHR Data Portal can allow a DC or DP to submit or respond to data requests via a user interface, automatic script execution, or other suitable mechanism. In another exemplary embodiment, the EHR Data Portal can maintain a DP or DC's tokens and/or financial information.

The server 102 can be implemented in hardware, software, or a suitable combination of hardware and software therefor, and may comprise one or more software systems operating on one or more servers, having one or more processors, with access to memory. Server(s) can include electronic storage, one or more processors, and/or other components. Server(s) can include communication lines, or ports to enable the exchange of information with a network and/or other computing platforms. Server(s) can also include a plurality of hardware, software, and/or firmware components operating together to provide the functionality attributed herein to server(s). For example, server(s) can be implemented by a cloud of computing platforms operating together as server(s). Additionally, the server can include memory 106.

Server(s) can include electronic storage, one or more processors, and/or other components. Server(s) can include communication lines, or ports to enable the exchange of information with a network and/or other computing platforms. Server(s) can also include a plurality of hardware, software, and/or firmware components operating together to provide the functionality attributed herein to server(s). For example, server(s) can be implemented by a cloud of computing platforms operating together as server(s).

Memory 106 can comprise electronic storage that can include non-transitory storage media that electronically stores information. The electronic storage media of electronic storage may include one or both of system storage that can be provided integrally (i.e., substantially non-removable) with server(s) and/or removable storage that can be removably connectable to server(s) via, for example, a port (e.g., a USIB port, a firewire port, etc.) or a drive (e.g., a disk drive, etc.). Electronic storage may include one or more of optically readable storage media (e.g., optical disks, etc.), magnetically readable storage media (e.g., magnetic tape, magnetic hard drive, floppy drive, etc.), electrical charge-based storage media (e.g., EEPROM, RAM, etc.), solid-state storage media (e.g., flash drive, etc.), and/or other electronically readable storage media. Electronic storage can include one or more virtual storage resources (e.g., cloud storage, a virtual private network, and/or other virtual storage resources). Electronic storage can store machine-readable instructions, software algorithms, information determined by processor(s), information received from server(s), information received from computing platform(s), and/or other information that enables server(s) to function as described herein. The electronic storage can also be accessible via a network connection.

Processor(s) 104 can be configured to provide information processing capabilities in server(s). As such, processor(s) can include one or more of a digital processor, an analog processor, a digital circuit designed to process information, an analog circuit designed to process information, a state machine, and/or other mechanisms for electronically processing information, such as FPGAs or ASICs. The processor(s) can be a single entity or include a plurality of processing units. These processing units can be physically located within the same device or represent the processing functionality of a plurality of devices operating in coordination or software functionality.

The processor(s) 104 can be configured to execute machine-readable instructions 108 or learning modules by software, hardware, firmware, some combination of software, hardware, firmware, and/or other mechanisms for configuring processing capabilities on processor(s). As used herein, the term “machine-readable instruction” may refer to any component or set of components that perform the functionality attributed to the machine-readable instruction component. This can include one or more physical processors during execution of processor readable instructions, the processor readable instructions, circuitry, hardware, storage media, or any other components.

The server 102 can be configured with machine-readable instructions 108 having one or more functional modules. The machine-readable instructions can be implemented on one or more servers, having one or more processors, with access to memory. The machine-readable instructions can be a single networked node, or a machine cluster, which can include a distributed architecture of a plurality of networked nodes. The machine-readable instructions can include control logic for implementing various functionality, as described in more detail below. The machine-readable instructions can include certain functionality associated with an EHR Data Portal, EHR Data API 110, EHR transaction blockchain API, a request analysis module 114, a data provisioning module 116, a royalty calculation module 118, and payment module 120. External databases 106 and external systems 140, as well as an EHR Data Blockchain client can also be implemented on one or more servers 102, having one or more processors 104, with access to memory 106.

Third party databases 106 can be operably coupled to the system components via the network 130. In one exemplary embodiment, the third-party databases 106 can include an electronic medical record system (EMR), an Electronic Patient Outcome Record (EPOR) database, pharmacy databases, a plurality of patient databases, a clinical database, a genomic database, a laboratory database, a disease database, a standardized drug database, a research database, and other suitable databases. In another exemplary embodiment, the third-party databases can function as archival nodes. The archival nodes can keep a real-time (sub-millisecond) encrypted copy of the distributed ledger 125. The archival node can provide fault tolerance and makes the contents of the distributed ledger 125 readily available to a host so that additional data processing, reporting, and analytics can be achieved. Instead of having to traverse the EHR Data API 110, the host can query its own machines to acquire the data. Any third party can host a drug archival node. In another exemplary embodiment, the archival node can provide data restoration to the distributed ledger 125. In another exemplary embodiment, the archival node can keep the older distributed ledger data accessible in a non-production system, so that the production distributed ledger can direct its full capabilities toward current transactions. In another exemplary embodiment, the distributed ledger can be transferred to the archival node once a distributed ledger transaction closes.

In one exemplary embodiment, the EHR Data API 110 can provide an interface that defines interactions between multiple software components. For example, EHR Data API 110 can define the kinds of calls or requests that can be made, how to make them, the data formats that should be used, the conventions to follow, and other suitable functionality. In another exemplary embodiment, the EHR Data API 110 can access and retrieve patient identifiable information (PII) and generate a non-patient-identifiable Single Purpose Patient II) (SPPID) for a particular patient, as well as timestamps.

In one exemplary embodiment, the EHR transaction blockchain API 112 can provide an interface that defines interactions between multiple software components. For example, EHR transaction blockchain API 112 can define the kinds of calls or requests that can be made, how to make them, the data formats that should be used, the conventions to follow, and other suitable functionality. In another exemplary embodiment, the transaction blockchain API 112 can be configured to store the timestamp, as well as particular, discrete data retrieved from the GPR for a patient, execute smart contracts, and control the execution of cryptocurrency transfers, among other functions.

In one exemplary embodiment, a request analysis module 114 can receive a request for data from an external system 140. In another exemplary embodiment, the request can be a query, having fields, parameters, or other relevant data. For example, a Data Consumer can generate a request for data related to a particular patient or patients having a common characteristic, among other relevant categories. In another exemplary embodiment, the request analysis module 114 can parse the request into discrete data elements to facilitate additional processing, such as field or parameter comparison or correlation. The request analysis module can append metadata to the request. For example, the metadata can include a unique identifier for the request, a unique identifier for the DC, a timestamp, or other relevant data.

In one exemplary embodiment, a data provisioning module 116 can transfer data to and from data providers via the server 102. Once a request is received, the data provisioning module 116 can transmit the query, or portions thereof, to one or more DPs to retrieve data responsive to the query. The data provisioning module can store the retrieved data in a patient record. In another exemplary embodiment, the record can be on the GPR or the server. In another exemplary embodiment, the data provisioning module 116 can format the query into a syntax requested by the DP. For example, the query syntax request can be identified in a DP profile stored in the EHR Data Portal.

In one exemplary embodiment, a royalty calculation module 118 can analyze the query responses and determine the proper royalty for each data request transaction. In one exemplary embodiment, a royalty percentage of a specific amount can be determined by the royalty calculation module 118 for the patient data. In another exemplary embodiment, a flat-fee processing fee can be paid to all parties involved in the query transaction. In another exemplary embodiment, the original data providers can also get paid for the data that they contributed. Since the patient owns its GPR, the patient can receive a royalty associated with the data transaction. The specific royalty amount can be predetermined based upon the cost of the service, cost of the data transfer, or other relevant metric. The royalty calculation module 118 can divide the royalty amount amongst the participating parties (including the patient), such that the DPs, the patient, and the Utility provider each can receive a percentage of the royalty, such that each party related to the transaction gets rewarded. The degree that each party gets rewarded can be an equal percentage (dividing the royalty by the number of parties involved in the transaction), biased such that the patient gets half of the royalty and the remaining parties equally split the remainder, or other suitable distribution. In another exemplary embodiment, the DP's royalty can be the largest percentage to entice DPs to share their data. In another exemplary embodiment, when a DP submits data to the Utility, the royalty calculation module 118 can generate and append metadata to the data to identify the DP, the patient (using a temporary patient identifier), and a timestamp, among other relevant data.

In one exemplary embodiment, the payment module 120 can determine if a smart contract was utilized during prescription filling and may result in the exchange of cryptocurrency (e.g., EHRCash™, Bitcoin®, etc.), utility tokens, vouchers, or other suitable notes, between the parties involved in the smart contract. For example, the exchange of EHRCash™ tokens can be immediate (sub-millisecond delay) and can eliminate the need for standard accounting processes (e.g., invoicing, statements, accounts receivable and accounts payable) in order to collect money from trading partners. In another exemplary embodiment, the payment module 120 can utilize stored wallet information to effect the cryptocurrency transactions. The modules described herein can be executed, called, or otherwise implemented via server, control logic, API, or other suitable means. The distributed ledger 125 can be an EHR Data blockchain implemented on one or more platforms, including BigChainDB, nChain, Ethereum, Hyperledger, R3, Ripple, EOS, or other suitable blockchain platform. The EHR Data blockchain can be a public blockchain, accessible to the general public, or a private blockchain, accessible only by those parties credentialed for access. External systems 140 can be operably coupled to the system components via the network 130. External systems 140 can include patient devices, pharmacy devices, payer devices, financial institution devices, insurance devices, or other suitable systems or devices. Such systems and devices can include smart phones, tablets, wearable devices, laptops, desktops, servers, appliances, or other suitable devices. In one exemplary embodiment, an external system 140 can be EHR-Data-BC-Client.

The aforementioned system components, APIs, and modules can be communicably coupled to each other via the Internet, intranet, system bus, or other suitable network 130. The communication can be encrypted, unencrypted, over a VPN tunnel, or other suitable communication means. The network 130 can be a WAN, LAN, PAN, or other suitable network. The network communication between the system components 102, 104, 106, 108, 110, and 112, can be encrypted using PGP, Blowfish, AES, 3DES, HTTPS, or other suitable encryption. The network communication can occur via application programming interface (API), health level 7 (HL7) standard, ANSI-X12, Ethernet, PCI, PCIe, InfiniBand, Wi-Fi, Bluetooth, or other suitable communication protocol.

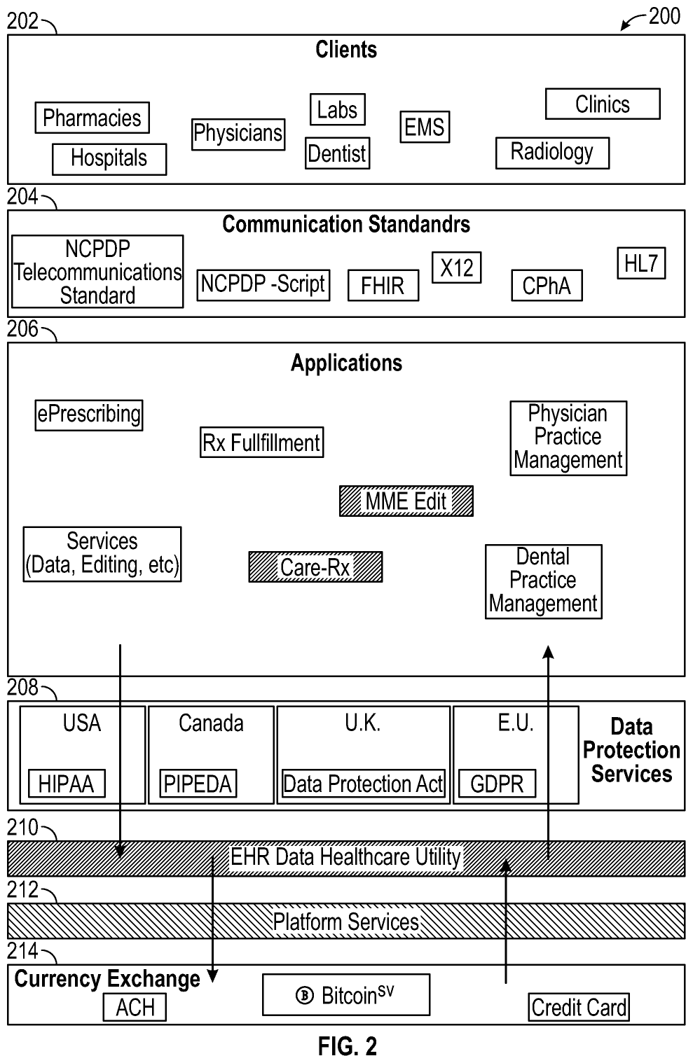

FIG. 2 illustrates a schematic view of an EHR data utility architecture 200, in accordance with one or more exemplary embodiments of the present disclosure. The EHR data utility architecture 200 can include clients 202, communication standards 204, applications 206, data protection services 208, an EHR Data Utility 210, platform services 212, and a currency exchange system 214.

In one exemplary embodiment, clients 202 can include Data Providers (DP) and Data Consumers (DC). DPs can provide patient data related to its particular industry. For example, pharmacies can provide patient prescription data, physicians can provide patient medical histories, laboratories can provide blood work test results, among others. DCs can utilize various patient data from DPs to provide better care to patients by considering a broader range of patient conditions and determining how those conditions can affect the DPs provision of services to the patient. Clients can include pharmacies, hospitals, physicians, laboratories, dentists, emergency medical services (EMS), radiology providers, insurance companies, and clinics, among other relevant industries.

In another exemplary embodiment, applications 206 can include various uses of patient data. For example, applications can include Electronic Prescription services, prescription fulfillment services, physician practice management, dental practice management, services (e.g., data, editing, etc.), and Morphine Milligram Equivalents (MME) edits, among other relevant applications.

In another exemplary embodiment, data protection services 208 can be varied based on country. For example, the EHR data utility system can establish policies and procedures for data handling that conform with, in the United States, the Health Insurance Portability and Accountability Act (HIPAA), in Canada, Personal Information Protection and Electronic Documents Act (PIPEDA), in the UK, the Data Protection Act, in the European Union, the General Data Protection Regulation (GDPR), and other relevant jurisdictional data privacy acts or provisions. In another exemplary embodiment, the policies and procedures can include receiving patient authorization for patient data disclosure, identification of the parties that can receive patient data, and identification of the types of patient data that can be disclosed, among other relevant policies and procedures.

The aforementioned system components, and their sub-components, can be communicably coupled to each other via the Internet, intranet, or other suitable network. The communication can be encrypted, unencrypted, over a VPN tunnel, or other suitable communication means. The Internet can be a WAN, LAN, PAN, or other suitable network. The network communication between the system components, and their sub-components, can be encrypted using PGP, Blowfish, Twofish, AES, 3DES, HTTPS, or other suitable encryption. The network communication can occur via communication standards 204, such as application programming interface (API), NCPDP telecommunications standard, NCPDP-Script, FHIR, ANSI-X12, CPhA, HL7, Ethernet, Wi-Fi, Bluetooth, PCI, PCI-Express, Fiber, or other suitable communication protocol, standard, or medium.

In one exemplary embodiment, the platform services 212 can provide access to internal and external databases and distributed ledgers. For example, platform services can incorporate modules that can be integrated to existing enterprise IT infrastructure to provide additional functionality (e.g., nChain® enterprise-grade blockchain solutions).

In one exemplary embodiment, the system can operably communicate with currency exchange systems 214, such as cryptocurrency (e.g., Bitcoin®), ACH services, banking services, wire transfer services, and other relevant currency exchange systems.

In one exemplary embodiment, in operation, the EHR Data Utility (Utility) can receive patient data from DPs that were authorized by the patient. The authorized patient data can be sold to a DC, who can incorporate the patient data into the analysis for providing its services (e.g., a pharmacy that receives multiomic data (data of different “omic” groups) for a patient to determine how quickly the patient will metabolize the data so the proper dosing can be determined). The DP will receive a royalty (payment) for the provision of its data to the DC. For example, a vaccination service (DP in this example) maintains patient data including the patient's vaccination data. The DC can receive the patient's vaccination data from the DP. The DC can then mix the vaccination data with its own patient demographic data. The DC could pay the DP for the vaccination data.

In one exemplary embodiment, a royalty percentage of a specific amount is determined by the EHR Data Utility for the vaccination data. The specific amount can be predetermined based upon the cost of the service, cost of the data transfer, or other relevant metric. The royalty amount can then be divided amongst the parties (including the patient), such that the DP′, the patient, and the EHR Data Utility provider receive a percentage of the royalty, such that each party related to the transaction gets rewarded. The degree that each party gets rewarded can be an equal percentage (dividing the royalty by the number of parties involved in the transaction), biased such that the patient gets half of the royalty and the remaining parties equally split the remainder, or other suitable distribution. The EHR Data Utility can be intended to be a low-cost solution so it can take a minimal royalty amount. The DP's royalty can be the largest percentage to entice DPs to share their data. In another exemplary embodiment, when a DP submits data to the Utility, the utility can generate and append metadata to the data to identify the DP, the patient (using a temporary patient identifier), and a timestamp, among other relevant data.

In another exemplary process, the Utility can receive and store a patient's prescription data on, a distributed ledger (e.g., a blockchain). Vaccination data can also be stored on the blockchain, thereby guaranteeing the immutability of the data and creating an opportunity to sell the vaccination and the prescription data. In another exemplary example, a DC (e.g., a drug company) can transmit a patient data request to the Utility for a listing of all of the women in the Utility database (e.g., EPOR) between the ages of 20 and 25 years old, that live in certain zip codes identified in the request, and that have been inoculated with a vaccine identified in the request. In order to respond with the requested information, multiple DPs patient data may be utilized to allow the filtering of patient data to allow the request to be fulfilled. A royalty can then be paid out to all of the DPs that contributed patient data that was used to fulfil the DC's request. Accordingly, multiple royalties can be paid out based on a single drug sale.

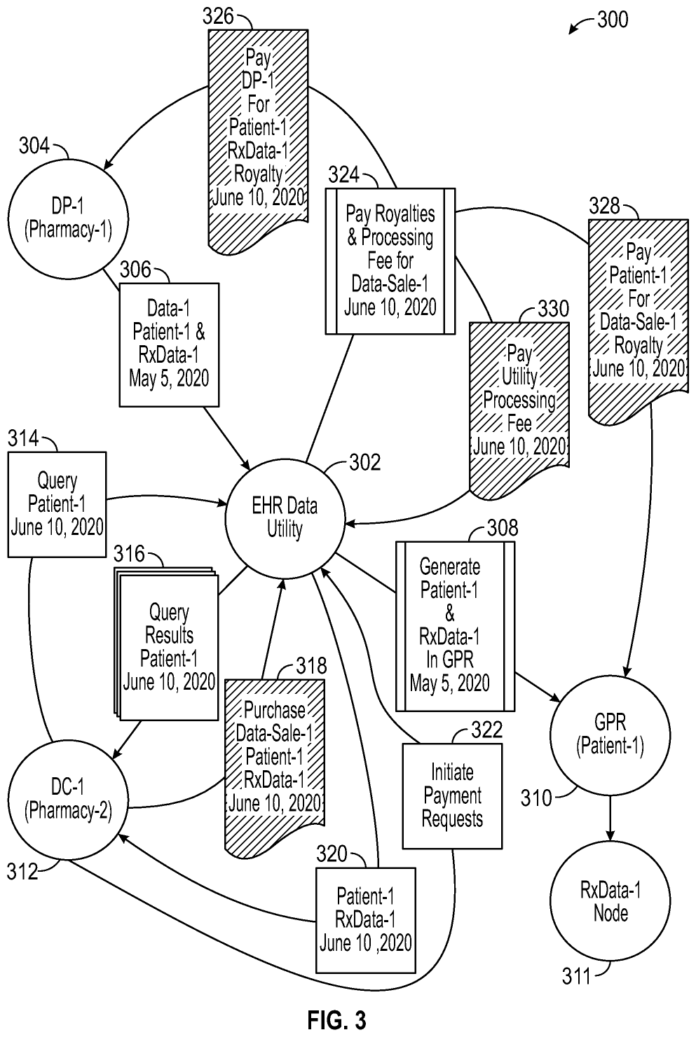

FIG. 3 illustrates a flowchart diagram exemplifying control logic 300 embodying features of a method of real-time patient data sale processing, in accordance with one or more exemplary embodiments of the present disclosure. The real-time patient data sale processing control logic 300 can be implemented as an algorithm on a processor, a server, a machine learning module, or other suitable system. The real-time patient data sale processing control logic 300 can be achieved via software, hardware, an application programming interface (API), a network connection, a network transfer protocol, HTML, DHTML, JavaScript, Dojo, Ruby, Rails, other suitable applications, or a suitable combination thereof.

The real-time patient data sale processing control logic 300 can leverage the ability of a computer platform to spawn multiple processes and threads by processing data simultaneously. The speed and efficiency of the real-time patient data sale processing control logic 300 can be greatly improved by instantiating more than one process. However, one skilled in the art of programming will appreciate that use of a single processing thread may also be utilized and is within the scope of the present disclosure. In one exemplary embodiment, the real-time patient data sale processing control logic 300 can instantiate various modules of the server. In another exemplary embodiment, the real-time patient data sale processing control logic 300 can be distributed across multiple servers, processors, appliances, or devices.

In one exemplary embodiment, the control logic 300 can provide for a DP-1 304 (e.g., Pharmacy-1) can submit Data-1 306 (e.g., Patient-1 and RxData-1) to the EHR Data Utility 302. In another exemplary embodiment, the control logic 300 can generate a Patient-1 record entry 308 to add the Data-1 306 received from the DP-1 304 to the GPR (Global Patient Record) 310 for Patient-1. If the Patient-1 GPR does not exist, the control logic 300 can create a new Patient-1 GPR. The GPR 310 can be viewed as the root of the patient hierarchy and the RxData-1 can be added as a patient node 311 to the GPR 310 hierarchy. The GPR 310 can include patient nodes 311 that include hierarchical information, such as demographics, pharmacy information, clinical information, multiomic information, and other relevant information. The GPR 310 data originates from DP-1 304, with a timestamp having a date and time (e.g., May 5, 2020 at 3:11 pm CDT), or other suitable date/time indicator. In another exemplary embodiment, a data provider (e.g., DP-1 304) can also be a data consumer (e.g., DC-1 312), depending on a particular situation, such as where the query originated from or where the relevant data resides.

In one exemplary embodiment, Data Consumer-1 (DC-1) 312 can query the control logic 300 for patient data (e.g., Patient-1 on Jun. 10, 2020). The query 314 can be transmitted from an external system 140 to the control logic 300 over an encrypted network. The query 314 can contain one or more fields and identify data stored in the GPR 310 or other suitable memory. For example, the patient may have gone to Pharmacy-1 to fulfill another prescription or transfer that prescription from another pharmacy. In another exemplary embodiment, the querying can occur on a blockchain, for example via one or more smart contracts. The querying process can be asynchronous such that when the initial patient GPRs are created they can be created on the blockchain. The requests 314 can then enter the blockchain queue. By writing directly to the blockchain, the control logic 300 doesn't have to wait to process the request 314.

In one exemplary embodiment, the control logic 300 can return a query result set 316 identifying patients having a GPR 310 record that matches one or more fields or parameters of the query request 314. For example, each field or parameter of the query can be correlated against a corresponding field in the GPR 310 for a plurality of patients. The control logic 300 can allow DC-1 312 to select a particular patient or patient group from the query result set 316. When Pharmacy-1 304 queries the control logic 300 to determine whether Patient-1 exists in the GPR 310, the Utility 302 can return a smaller query result subset, such that all the patients that match that criteria can be returned. In another exemplary embodiment, each field or parameter in the query 314 can be used to further filter the query results 316. For example, additional fields or parameters of the query 314 can be correlated against a corresponding field in the query result subset.

In one exemplary embodiment, once the query results are reviewed, DC-1 312 (e.g., Pharmacy-2) can purchase patient data (e.g., Patient-1 demographics and RxData-1) using the control logic 300 via a user interface on the Utility 302 to generate a data purchase request 318. In another exemplary embodiment, the DC-1 312 can issue a purchase request 318 for that particular patient data or a portion thereof. As an example, DC-1 312 may want to purchase only the Patient-1 demographics, because DC-1 312 may not need the current prescription information to run the particular patient analysis. In another example, DC-1 312 can purchase all available Patient-1 data. The control logic 300 can allow a DC to identify a subset of the full patient data set for a patient for purchase, or the full patient data set via a user interface. For example, the control logic 300 can provide for the selection for one or more fields or parameters of patient data for purchasing via a user interface on Utility 302.

In one exemplary embodiment, the control logic 300 can return patient data 320 (e.g., Patient-1 demographics and RxData-1) to IDC-1 312. The control logic 300 can return all the data related to a patient, or a portion thereof. Once the patient data 320 is returned to Pharmacy-2 312, Pharmacy-2 312 can initiate a payment request 322 using the control logic 300. The control logic 300 can automatically identify the recipients of a payment request by maintaining a list of the parties that provide patient data for the transaction. In another exemplary embodiment, the control logic 300 can transfer an electronic payment from one party's (e.g., Pharmacy-2) account to another party's (e.g., Pharmacy-1) account using the financial data stored in the party's EHR Data Portal profile maintained on the Utility 302. The control logic 300 can maintain a record for each client that includes financial information for the client, such that payments can be transferred by the Utility 302 via the platform services 212 to one or more currency exchange systems 214. In another exemplary embodiment, the client record maintained on the Utility 302 can function as a digital currency wallet. In another exemplary embodiment, once a payment request has been generated, the royalties can be determined.

In one exemplary embodiment, the control logic 300 can calculate and process 324 a royalty (e.g., a royalty to DP-1 at 326, a royalty to Patient-1 at 328, and a royalty or processing fee to the transaction processor at 330). In another exemplary embodiment, the royalty to be paid to the patient can be associated with the patient's GPR. In another exemplary embodiment, a flat-fee processing fee can be paid to all parties involved in the query transaction. In another exemplary embodiment, the original data providers can get paid for the data that they contributed. Since the patient owns its GPR, the patient can receive a royalty associated with the data transaction. In another exemplary embodiment, the control logic 300 can effect the transfer of data and payment of royalties and processing fees via a smart contract on a blockchain via the Utility 302. The platform services 212 can be an API that can communicate directly with a digital currency exchange 214, such as the Bitcoin SV blockchain. The transaction on the digital currency exchange (e.g., Bitcoin SV blockchain) can transfer the identified number of tokens or currency amount from the DC-1 312 account to the DP-1 304 account. In another exemplary embodiment, the currency exchange can be via credit or debit card transfer. In another exemplary embodiment, the payment can be effectuated via bank-to-bank transfer, or other suitable form of payment. The control logic 300 provides the technological advantage of providing a single source of truth and rewarding people or companies for the use of their data.

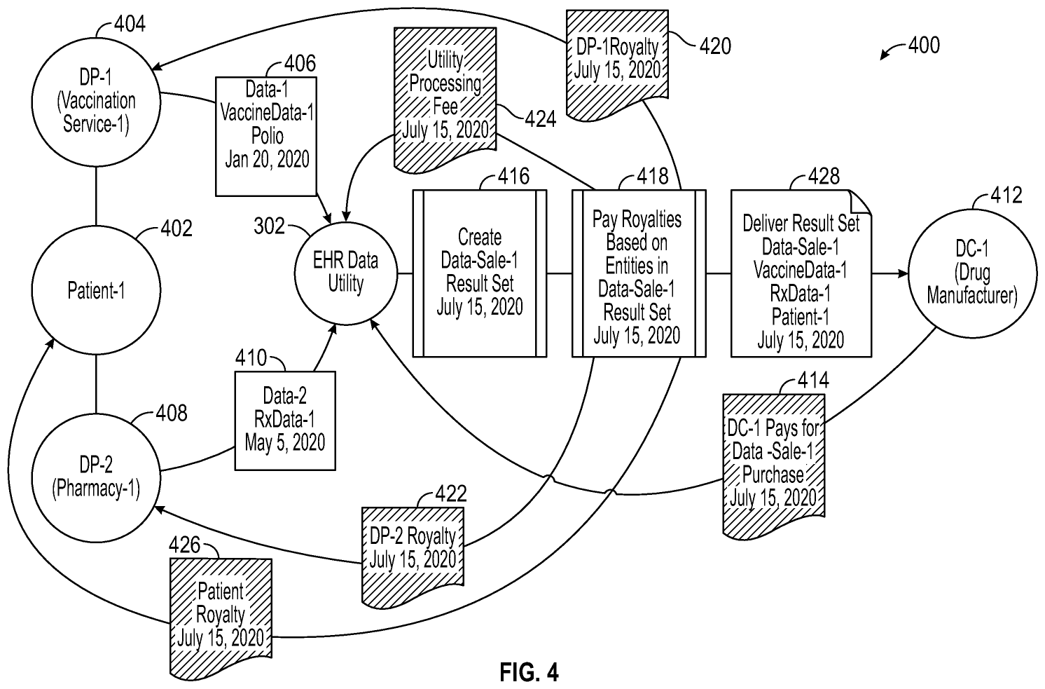

FIG. 4 illustrates a flowchart diagram exemplifying control logic 400 embodying features of a method of ad hoc patient data purchase processing, in accordance with one or more exemplary embodiments of the present disclosure. The ad hoc patient data purchase processing control logic 400 can be implemented as an algorithm on a processor, a server, a machine learning module, or other suitable system. The ad hoc patient data purchase processing control logic 400 can be achieved via software, hardware, an application programming interface (API), a network connection, a network transfer protocol, HTML DHTML, JavaScript, Dojo, Ruby, Rails, other suitable applications, or a suitable combination thereof.

The ad hoc patient data purchase processing control logic 400 can leverage the ability of a computer platform to spawn multiple processes and threads by processing data simultaneously. The speed and efficiency of the ad hoc patient data purchase processing control logic 400 can be greatly improved by instantiating more than one process. However, one skilled in the art of programming will appreciate that use of a single processing thread may also be utilized and is within the scope of the present disclosure. In one exemplary embodiment, the ad hoc patient data purchase processing control logic 400 can instantiate various modules of the server. In another exemplary embodiment, the ad hoc patient data purchase processing control logic 400 can be distributed across multiple servers, processors, appliances, or devices.

In one exemplary embodiment, the control logic 400 can provide for DP-1 404 (Vaccination Service-1) to add vaccination data 406 for Patient-1 402 to the EHR Data Utility 302 and append a timestamp (e.g., on Jan. 20, 2020) of the transmission or transaction to the data. For example, vaccine data 406 can be included in a patient's GPR, as well as the date a vaccine (e.g., polio vaccine, VaccineData-1) was administered (e.g., Jan. 20, 2020). In another exemplary embodiment, the ad hoc system can be similar to a batch operation with certain real-time implementations.

In one exemplary embodiment, DP-2 408 (Pharmacy-1) can request access via the control logic 400, to a patient's GPR. For example, on May 5, 2020, DP-2 (Pharmacy-1) fills a prescription for Patient-1 402. The prescription fulfilment 410 can be added to the GPR for the patient, maintained by the EHR Data Utility 320.

In one exemplary embodiment, the control logic 400 can receive a Data-Sale-1 purchase request from DC-1 412 (Drug Manufacturer)(e.g., on Jul. 15, 2020). A data consumer can request a patient data report that matches identified criteria identified in the purchase request. The patient data report can be requested via the EHR Data Portal. The DC-1 412 can pay for the Data-Sale-1 purchase request 414 via the Utility 302, which can be operably coupled to a currency exchange. In one exemplary embodiment, the control logic 400 can query a database for patients matching the data sales criteria and retrieve patient data to generate a Data-Sale-1 result set 416. In one exemplary embodiment, the database can be queried via a smart contract operably coupled to the database. The database can be local to the Utility 302 or a remote third-party database. The control logic 400 can generate the result set 416 for the DC-1 412 related to the Data-Sale-1 414. For example, the control logic 400 can generate a record to maintain a listing of all data responsive to the query. The control logic 400 can compare or correlate one or more fields or parameters of the data sales criteria with the database fields or parameters of patient records (e.g., GPR patient records) to generate the result set with the patient data meeting the criteria. In another exemplary embodiment, the criteria can include demographic parameters (e.g., race, age, weight, etc.). In another exemplary embodiment, the criteria can include pharmaceutical parameters (e.g., prescriptions, dosage, frequency, etc.). In another exemplary embodiment, the criteria can include laboratory parameters (e.g., cholesterol level, blood type, lab work values, etc.). In another exemplary embodiment, the criteria can include genomic parameters (e.g., metabolic information, etc.). In another exemplary embodiment, the criteria can include personality parameters (e.g., wake time, bed time, workout frequency, computer usage, etc.).

In one exemplary embodiment, the control logic 400 can pay a processing fee or royalty fee 418 to itself 424, and a royalty payment to the other parties involved in the data transaction (e.g., DP-1 at 420. DP-2 at 422, and Patient-1 at 426). In another exemplary embodiment, the control logic 400 can facilitate the payment of a flat-fee processing fee to all parties involved in the query transaction. The original data providers can get paid for the data that they contributed. Since the patient owns its GPR, the patient gets a royalty associated with the data on the date of sale. In another exemplary embodiment, the control logic 400 can effect the transfer of data and payment of royalties and processing fees via a smart contract on a blockchain. The platform services can be an API that can create a digital/crypto currency transaction directly on a blockchain, such as the Bitcoin SV blockchain. The transaction on the Bitcoin SV blockchain can transfer the identified number of tokens from the DC-1 412 account to DP-1 404's account. In another exemplary embodiment, the currency exchange can be via credit or debit card transfer. In another exemplary embodiment, the payment can be effectuated via bank-to-bank transfer, or other suitable form of payment. The control logic 400 provides the technological advantage of providing a single source of truth and the rewarding people or companies for the use of their data.

In one exemplary embodiment, control logic 400 can deliver the Data Sale-1 Result Set 428 to DC-1 412. For example, the delivered result set 428 can include VaccineData-1 and RxData-1 for Patient-1 402. In another exemplary embodiment, the result set can be sent after the payment of all fees and royalties related to the data transaction.

Advantageously, the Utility is a low-cost solution for healthcare systems that can provide storage for their patient data. The Utility can be cloud-based and highly scalable. Ultimately clients would have no reason for their own data center because the Utility would have the latest data. Given the broad swath of the aggregated data, the client's data could be incomplete just after the Utility data is accessed, as a patient may have taken additional treatment. The patient data may not change but it could be incomplete as maybe more prescriptions are added for a patient. The Utility can also reduce pharmacists' efforts doing menial tasks, such as entering patient addresses, phone numbers, insurance information, and capturing the patient's prescription history.

In one exemplary embodiment, the patient data provided can include all patient data, or only a small subset. In another exemplary embodiment, the royalty to be paid to the parties can be priced according to the portion of the patient's GPR received.

Persons skilled in the art will readily understand that these advantages (as well as the advantages indicated in the summary) and objectives of this system would not be possible without the particular combination of computer hardware, control logic, and other structural components and mechanisms assembled in this inventive system and described herein. It will be further understood that a variety of programming tools, known to persons skilled in the art, are available for implementing the control of the features and operations described in the foregoing disclosure. Moreover, the particular choice of programming tool(s) may be governed by the specific objectives and constraints placed on the implementation selected for realizing the concepts set forth herein and in the appended claims.

The description in this patent document should not be read as implying that any particular element, step, or function can be an essential or critical element that must be included in the claim scope. Also, none of the claims can be intended to invoke 35 U.S.C. § 112(f) with respect to any of the appended claims or claim elements unless the exact words “means for” or “step for” are explicitly used in the particular claim, followed by a participle phrase identifying a function. Use of terms such as (but not limited to) “mechanism,” “module,” “device,” “unit,” “component,” “element,” “member,” “apparatus,” “machine,” “system,” “processor” “processing device,” or “controller” within a claim can be understood and intended to refer to structures known to those skilled in the relevant art, as further modified or enhanced by the features of the claims themselves, and can be not intended to invoke 35 U.S.C. § 112(f).

The disclosure may be embodied in other specific forms without departing from the spirit or essential characteristics thereof. For example, each of the new structures described herein, may be modified to suit particular local variations or requirements while retaining their basic configurations or structural relationships with each other or while performing the same or similar functions described herein. The present embodiments are therefore to be considered in all respects as illustrative and not restrictive. Accordingly, the scope of the inventions can be established by the appended claims rather than by the foregoing description. All changes which come within the meaning and range of equivalency of the claims are therefore intended to be embraced therein. Further, the individual elements of the claims are not well-understood, routine, or conventional. Instead, the claims are directed to the unconventional inventive concept described in the specification.

What is claimed is:

1. A method of providing real-time patient data sale processing, the method comprising the steps of:
generating, via a processor, a global patient record for a first patient:
receiving a query request having one or more parameters for patient data from a data consumer over an encrypted network;
correlating, via an EHR Data utility, the one or more parameters with a plurality of global patient records stored in a database to find matching patient data;
returning the matching patient data from one or more data providers to the data consumer, and paying a royalty to the one or more data providers, the utility and the first patient.

2. The method of claim 1, wherein the query parameters include demographic data.

3. The method of claim 1, wherein the query parameters include pharmaceutical data.

4. The method of claim 1, wherein the query parameters include laboratory data.

5. The method of claim 1, wherein the query parameters include mutiomic data.

6. The method of claim 1, wherein the query request is stored in a distributed ledger.

7. The method of claim 1, further comprising automatically identify the recipients of a payment request by storing a list of the parties that provide patient data included in the matching patient data.

8. The method of claim 1, wherein the royalty is a flat-fee processing fee paid to all parties involved in the query transaction.

9. The method of claim 1, wherein a royalty percentage is calculated based upon the amount of data contributed.

10. The method of claim 1, wherein the paying of royalties is executed via a smart contract on a blockchain.

11. A method of providing ad-hoc patient data purchase processing, the method comprising the steps of:
storing patient-related data received from a data provider in a global patient record database;
receiving a data sale request, having one or more data sales criteria, from a data consumer;
querying the database for patient data matching the data sales criteria;
generating a data sale result set having patient data that matches the data sale request; and
facilitating settlement between the data consumer and the data provider by paying a royalty to the parties that contributed data to the data sale result set.

12. The method of claim 11, wherein the criteria include demographic data.

13. The method of claim 11, wherein the criteria include pharmaceutical data.

14. The method of claim 11, wherein the criteria include laboratory data.

15. The method of claim 11, wherein the criteria include mutiomic data.

16. The method of claim 11, wherein the criteria is stored in a distributed ledger.

17. The method of claim 11, further comprising automatically identify the recipients of a data sale purchase by storing a list of the parties that provide patient data included in the result set.

18. The method of claim 11, wherein the royalty is a flat-fee processing fee paid to all parties involved in the query transaction.

19. The method of claim 11, wherein a royalty percentage is calculated based upon the amount of data contributed.

20. The method of claim 11, wherein the paying of royalties is executed via a smart contract on a blockchain.
